(19) United States
(12) Patent Application Publication
(10) Pub. No.: US 2013/0072781 A1
(43) Pub. Date: Mar. 21, 2013

(54) AUTOMATIC AND SEMI-AUTOMATIC PARAMETER DETERMINATIONS FOR MEDICAL IMAGING SYSTEMS

(71) Applicants: Jon Charles Omernick, Wauwatosa, WI (US); Daniel Mabini, Waukesha, WI (US); Teri Fischer, Waukesha, WI (US)

(72) Inventors: Jon Charles Omernick, Wauwatosa, WI (US); Daniel Mabini, Waukesha, WI (US); Teri Fischer, Waukesha, WI (US)

(73) Assignee: General Electric Company, Schenectady, NY (US)

(21) Appl. No.: 13/237,787

(22) Filed: Sep. 20, 2011

(51) Int. Cl. A61B 5/055; A61B 8/00; G06K 9/00; A61B 5/05

(52) U.S. Cl. 600/410; 600/407; 600/437; 382/131

(57) ABSTRACT

A medical imaging analysis method includes the step of receiving parameter data from an imaging component. The parameter data corresponds to at least two imaging operations and encodes at least two parameter sets corresponding to the at least two imaging operations. The method further includes the step of comparing the at least two parameter sets to identify a grouping that repeats between the parameter sets a number of times that exceeds a first threshold, an implemented change to a default parameter that repeats between the parameter sets a number of times that exceeds a second threshold, or a combination thereof.

### BACKGROUND OF THE INVENTION

The subject matter disclosed herein generally relates to medical imaging systems and, more particularly, to automatic and semi-automatic parameter determinations for these medical imaging systems.

A wide range of tissues may be imaged in a medical field through the use of various types of imaging systems. Many different types of imaging systems have been developed and refined, including X-ray systems, which have moved from film-based systems to digital X-ray. Other important modalities include magnetic resonance imaging systems, computed tomography imaging systems, ultrasound systems, positron emission tomography systems, X-ray tomosynthesis systems, and so forth. In all of these imaging systems, image data is acquired and stored for later processing and eventual image reconstruction. In a typical setting, reconstructed images are most often presented to a radiologist or other physician or clinician for use in rendering care.

All of these imaging systems typically include a user interface that enables a user to specify parameters of the imaging operation that are utilized by the imaging device associated with the given modality to facilitate data acquisition in the desired manner. Upon system installation, a set of default parameters is typically preloaded, and these default parameters provide the user with a baseline when determining the appropriate parameters for the given application. However, while these default parameters may simplify the process of setting up the imaging system for image acquisition, many inefficiencies still exist when the user interfaces with the imaging system to define desired parameters. For example, in some instances, the user may repeatedly change the default parameters for a series of imaging operations if the default parameters do not meet the operator's needs.

### BRIEF DESCRIPTION OF THE INVENTION

In accordance with aspects of the present techniques, a medical imaging analysis method includes the step of receiving parameter data from an imaging component. The parameter data corresponds to at least two imaging operations and encodes at least two parameter sets corresponding to the at least two imaging operations. The method also includes the step of comparing the at least two parameter sets to identify a grouping that repeats between the parameter sets a number of times that exceeds a first threshold, an implemented change to a default parameter that repeats between the parameter sets a number of times that exceeds a second threshold, or a combination thereof.

The techniques also offer a medical imaging system includes an imager adapted to generate image data indicative of a region of interest in a patient and an operator interface adapted to receive one or more operator selections corresponding to parameters of an imaging operation to be performed by the imager. The medical imaging system also includes control circuitry coupled to the imager and the operator interface and adapted to control the imager in accordance with the operator selections to acquire signals that may be converted to the image data. The medical imaging system also includes data processing circuitry adapted to receive a parameter set containing the operator selections from the operator interface and to analyze the received parameter set via comparison with a previously received parameter set to identify the frequency of one or more changes to one or more default parameters.

In accordance with another aspect, a medical imaging system includes an operator interface adapted to receive operator selections corresponding to a desired exam protocol grouping for an imaging operation to be performed by a medical imaging device. The medical imaging system also includes data processing circuitry adapted to receive a parameter set containing the operator selections from the operator interface, to compare the desired exam protocol grouping with at least one previously received exam protocol grouping to identify one or more protocol groupings that occur at a frequency exceeding a threshold, and to recommend establishment of the identified protocol groupings as favorite groups.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of an imaging system capable of identifying frequently utilized parameters for an imaging modality suitable for imaging of a patient;

FIG. 2 is a flow diagram illustrating an exemplary method for performing data analysis and providing parameter recommendations for the imaging system of FIG. 1;

FIG. 3 is a flow diagram illustrating an exemplary method for identifying frequently utilized exam protocol groupings;

FIG. 4 is a flow diagram illustrating an exemplary method for identifying parameters of an exam protocol that a user frequently changes from a default value;

FIG. 5 is a flow diagram illustrating an exemplary method for identifying a parameter of a step of an exam protocol that a user frequently changes from a default value;

FIG. 6 is an diagrammatical overview of an exemplary imaging system that may be employed in connection with the methods summarized in the preceding figures;

FIG. 7 is diagrammatical overview of an exemplary digital X-ray system that may be employed in connection with the methods summarized in the preceding figures;

FIG. 8 is an overview of an exemplary magnetic resonance imaging system that may be employed in connection with the methods summarized in the preceding figures;

FIG. 9 is a diagrammatical overview of an exemplary computed tomography imaging system that may be employed in connection with the methods summarized in the preceding figures;

FIG. 10 is an exemplary positron emission tomography imaging system that may be employed in connection with the methods summarized in the preceding figures; and

FIG. 11 illustrates an exemplary operator interface that may enable selection of exam protocols in connection with the methods summarized in the preceding figures.

### DETAILED DESCRIPTION OF THE INVENTION

As described in detail below, methods and systems are provided for determining one or more desirable changes to a default setting of an imaging system based on operator usage. For example, in some embodiments, the imaging system may include circuitry that monitors each imaging operation to identify which exam protocols are frequently implemented together by the operator. Once identified, the imaging system may recommend establishment of a favorite group that includes the exam protocols that are frequently utilized together by the operator. For further example, in additional embodiments, the imaging system may monitor changes made by the operator to one or more default parameters (e.g., magnification, dose level, etc.) in multiple imaging operations. In this way, the imaging system may identify operator usage patterns and utilize these patterns to recommend changes to the default parameters in accordance with prior usage. The foregoing features of presently disclosed embodiments may offer advantages over systems in which the default parameter values and favorite groupings are inflexible or rely on manual reprogramming in order to make changes to the default settings. For instance, these features may reduce or eliminate the need for an operator to repeatedly update parameter values or establish the frequently utilized desired groupings.

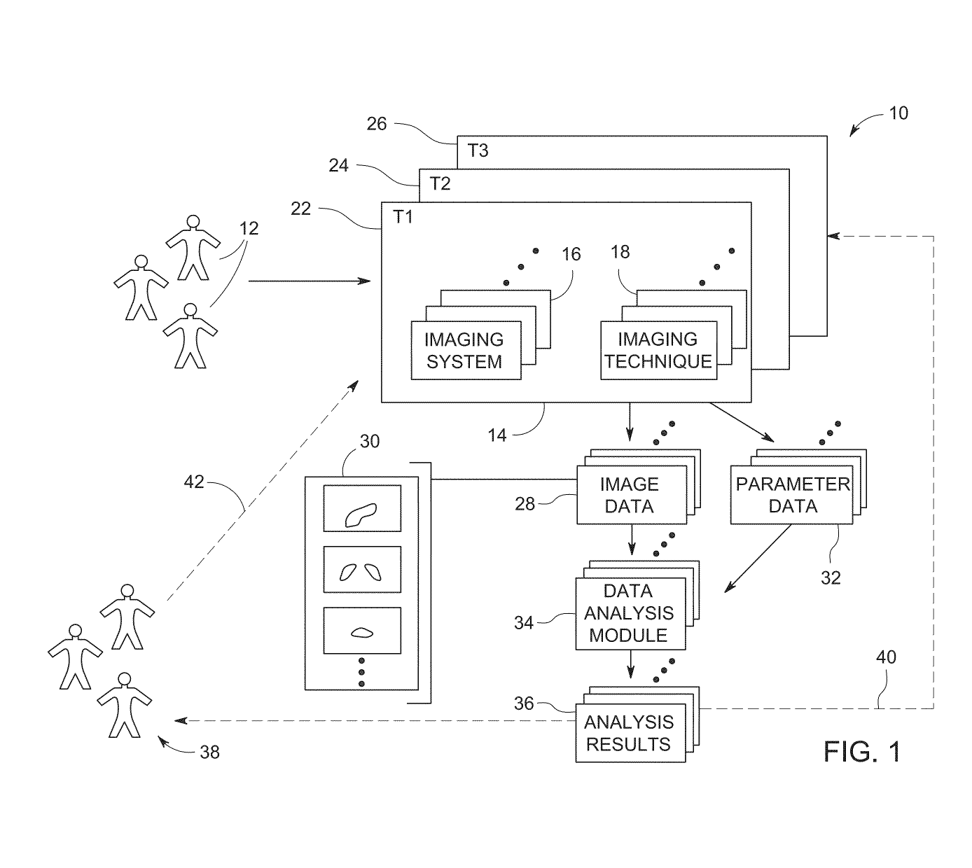

Turning now to the drawings, FIG. 1 is a diagrammatical overview of an imaging system 10 suitable for imaging of a patient and capable of identifying frequently utilized parameters across a variety of imaging operations. The system 10 is based upon use of one or more imaging technologies that are used to collect data relating to internal tissues, organs, structures, and so forth in a plurality of patients 12. In accordance with the technique, one patient 12 is subjected to an imaging procedure at a time. Accordingly, an imaging component 14 is employed to collect data for later analysis and, if desired, image reconstruction. That is, the imaging component 14 may collect image data indicative of a region of interest in the patient 12 as well as data regarding the parameters and groupings selected by an operator for the imaging operation.

The imaging component 14 will typically include one or more imaging systems 16 used in conjunction with one or more imaging techniques 18. As described in more detail below, the imaging systems 16 may include a variety of imaging modalities, including, but not limited to digital X-ray systems, computed tomography (CT) systems, magnetic resonance imaging (MRI) systems, positron emission tomography (PET) systems, ultrasound systems, X-ray tomosynthesis systems, and so forth. As appreciated, such systems may be considered to be different imaging “modalities” by virtue of their use of different imaging physics. Additionally, it should be noted that the default parameters and groupings that are monitored over the course of operator usage may vary in accordance with the imaging modality being employed. Nevertheless, regardless of the imaging modality employed, the imaging systems 10 disclosed herein are configured to monitor and trend the operator usage of the system.

The imaging techniques 18 may be considered different techniques that may be used on a single type of imaging system or modality system. Such techniques may include particular types of image data acquisition, specific types of data processing, various types of patient positioning and patient control, and so forth. By way of example only, within the X-ray field, imaging techniques may include various patient positioning and orientation to create projections that best show anatomies of interest. In the computed tomography imaging arena, various types of scans may be performed as imaging techniques. Such scans may include helical scans wherein a table is displaced in a scanner, various types of volumetric scanning, scout-mode scanning, as well as techniques for identifying various data windows of interest for image analysis and reconstruction. In the magnetic resonance imaging field, such techniques may include various pulse sequence descriptions that are specifically designed to create magnetic resonance echoes from various types of tissues, fluids, contrast agents, and the like.

As illustrated in FIG. 1, the imaging component 14, including the imaging systems 16 and imaging techniques 18 may be employed at different times, as indicated by blocks 22, 24, and 26. It should be noted that the times 22, 24 and 26 may reflect collection of image data on different patients at different times with the same or different operators to facilitate both the monitoring and trending of parameters utilized for multiple patients as well as to enable monitoring and trending of the preferences of a particular operator. Nevertheless, this acquisition of images at different times facilitates the comparison of parameters and/or groupings specified for acquisition of each image or set of images. For example, imaging at different times may illuminate parameter patterns (e.g., certain settings are typically employed for pediatric patients) or enable the identification of parameters that are frequently changed from the default value.

Moreover, it should be noted that the times 22, 24, and 26 may be remote from one another, such as removed from one another by days, weeks, months or even years. In other contexts, however, the times will be very close in proximity, such as for acquiring image data and processing the data during a procedure. As such, the default parameters and groupings may be trended and updated more or less frequently during use. Further, as more data becomes available over the course of additional operator usage of the imaging system over time, the default parameter values and groupings may be continuously updated to reflect, for example, patterns that emerge with continued monitoring and trending.

The imaging component 14 will generate image data that is stored for immediate or later processing, as indicated at reference numeral 28 in FIG. 1. The image data may be stored in accordance with conventional techniques, such as in memory circuits of the imaging system itself, or in departmental or hospital storage systems, archive systems and so forth. The image data will typically include data encoding picture elements (pixels) or volume elements (voxels) either in a processed form, a raw form or a semi-processed form. In all of these cases, however, the image data will include data that can be analyzed for evaluation and, in most cases, eventual reconstruction of an image of target anatomies, as indicated generally by reference numeral 30 in FIG. 1.

The imaging component 14 will also generate parameter data 32 corresponding to each of the acquisition blocks 22, 24, and 26 and capable of being similarly analyzed or stored for further processing. The parameter data 32 may include operator selections specific to each of the imaging operations performed in blocks 22, 24, and 26. For example, the parameter data 32 may include one or more exam protocol groupings selected by the operator for use during the imaging operation. In one embodiment, the operator selected grouping may be, for example, a series of desired exams performed on a particular region of the patient (e.g., a view of the sternum grouped with a view of the ribs above the diaphragm). For further example, the parameter data 32 may include operator-implemented changes to global protocol parameters and/or step parameters. For instance, in one embodiment, the parameter data 32 may include changes made to the default field of view, magnification, and dose level for the given medical imaging procedure. Again, the parameter data 32 may include multiple sets of this type of data, suitable for further analysis (e.g., comparison) by other system components.

Additionally, one or more data analysis modules 34 may be provided within the imaging system itself or at another remote location within, for example, a different area of a medical institution. Depending upon the given application and the type of data, the data analysis modules 34 may be considered to include one or more appropriately programmed general purpose or application-specific computers with suitable firmware or software. In general, the data analysis modules 34 permit the raw or processed image data 28 and parameter data 32 from the imaging system to be analyzed as desired for the given application. The data analysis modules may be of different types, depending upon the data type, the analysis to be performed, and the imaging system or even the imaging technique used to generate the image data and the parameter data.

One function of the data analysis modules 34 may be to process the received image data to provide image and analysis results 36, for example, by reconstructing a portion of the patient's anatomy. These results and analyses may be rendered immediately, that is, during or immediately subsequent to the image data acquisition, such as for specific on-going procedures. In other cases, the image data and analysis results may be provided subsequently, such as for diagnosis and planning of treatment, or for following up on treatment. In certain cases the analysis results may be provided in forms other than image-based forms, including reports, textual summaries, and the like. In many situations, the results may be separately stored for remote transmission, printing, archiving, and so forth.

Another function of the data analysis modules 34 may be to process the received parameter data 32. For example, in particular embodiments, the analysis modules 34 may monitor the parameter data 32 to identify a trend in the changes an operator makes to one or more parameters of the imaging operations. Based on the identified trend, the analysis module 34 may determine a suitable recommendation for a change to the default value of the parameter for which the trend was identified and may output this recommendation as a result 36 of the analysis. For further example, in some embodiments, the analysis module 34 may analyze the selected protocols in each of the blocks 22, 24, and 26 to identify one or more protocol groupings that occur at a frequency that exceeds a preset threshold. In instances in which a series of protocols are grouped by the operator a number of times that exceeds a predetermined number of times or are grouped in a preset percentage of the monitored imaging operations (e.g., protocols are grouped in approximately 80% of the imaging operations performed), this grouping of protocols may be recommended for implementation as a favorite grouping. The favorite grouping may then be chosen by the operator for future operations without having to individually select each of the protocols in the group.

Ultimately, the results 36 of the data analysis performed on the image data 28 and/or the parameter data 32 may be provided to medical professionals, as indicated by reference numeral 38, and/or to the imaging components 14, as indicated by arrow 40. For example, the recommendations for favorite groupings or changes to default parameter values determined by the analysis module 34 may be provided to medical professionals 38, such as radiologists, specialized physicians, primary physicians, clinicians, and other health care professionals, for acceptance or rejection. That is, in some embodiments, before being implemented, the determined recommendations are communicated to the medical professional 38, and, if desired, the medical professional 38 accepts the recommendations, as indicated by arrow 42. Alternatively, in certain embodiments, once the recommendations are identified and exported as analysis results 36, the system may be automatically updated to reflect the identified groupings and/or changes to the parameter values, as indicated by arrow 40. As previously noted, the recommendations based on the performed analysis may be provided both locally and immediately, such as during a procedure, or may be provided remotely and at subsequent times. In general, however, the information is provided for the purpose of updating the default parameter values and/or the favorite groupings in accordance with operator usage to reduce or eliminate the time necessary for the operator to set up the imaging system for the desired use.

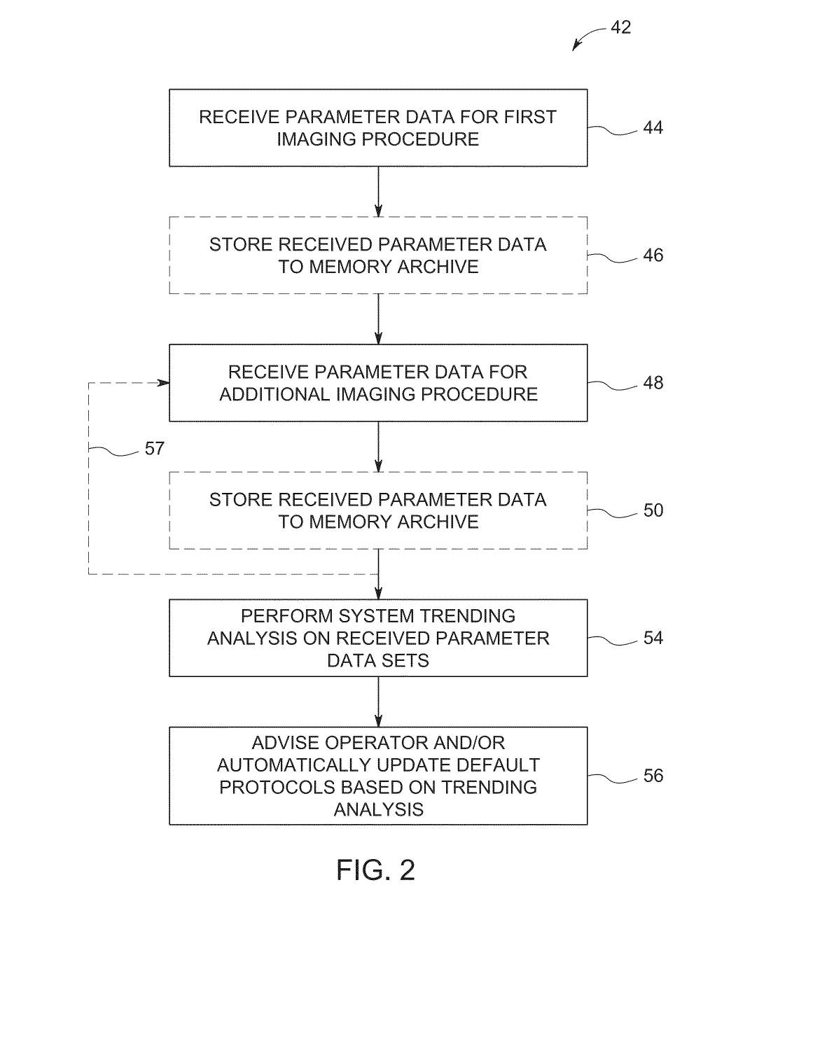

FIG. 2 is a flow diagram illustrating an exemplary method 42 that may be employed by the analysis module 34 of FIG. 1 to perform data analysis on the parameter data and to provide parameter recommendations in accordance with a disclosed embodiment. The method 42 includes receiving the parameter data for a first imaging procedure (block 44) and optionally storing the received parameter data to a memory archive (block 46). Likewise, parameter data for an additional imaging procedure is also received (block 48) and, if desired, stored to the memory archive (block 50). As indicated by the broken line 52 in FIG. 2, parameter data may be received and optionally stored multiple times, for example, over a predefined usage period.

Once the parameter data for the desired usage period is received, a trending analysis is performed on the received data sets (block 54). For example, one or more trends in changes to default parameter values may be identified across the data sets. For further example, one or more trends may be identified in the grouping of exam protocols chosen by the operator. Based on this system trending analysis, the method 42 includes advising the operator or automatically updating the system defaults to reflect the identified usage trends over the usage period (block 56). In this way, presently disclosed embodiments may be capable of automatically updating or suggesting updates to the default settings of the imaging system based on an analysis of past system usage.

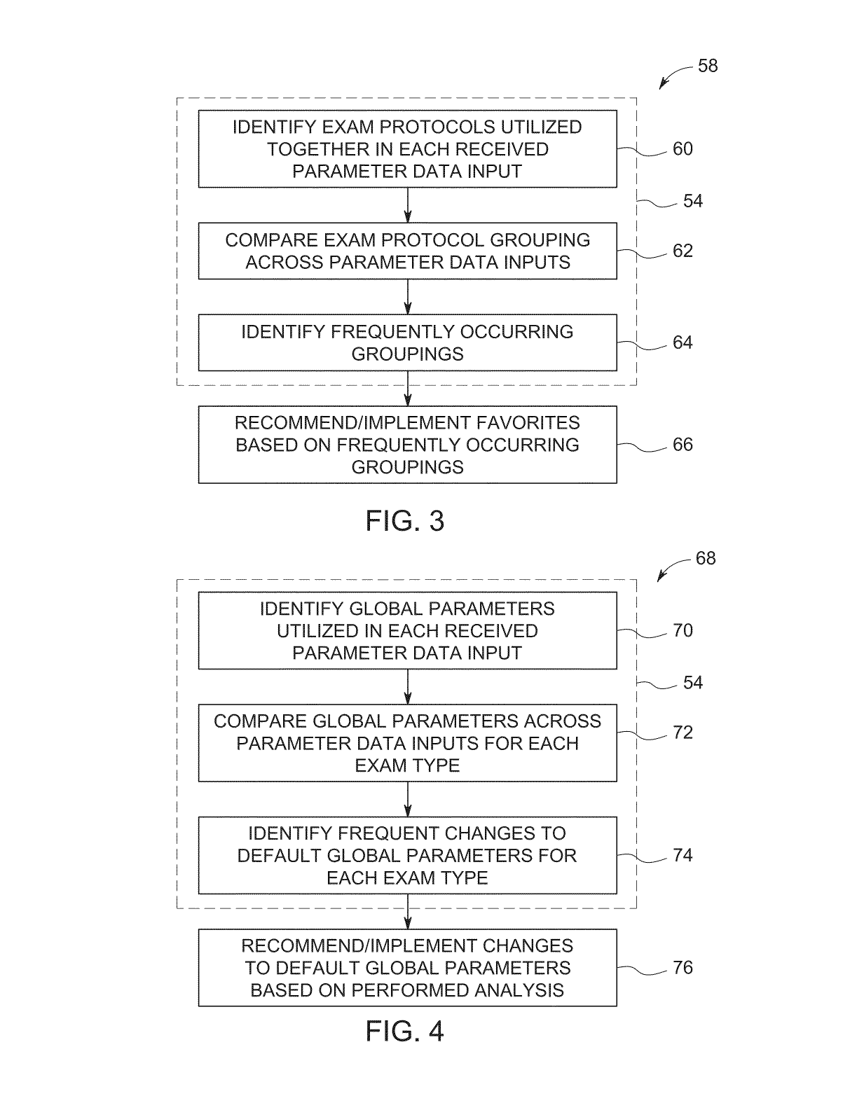

More specifically, FIG. 3 illustrates a method 58 for system trending of exam protocols to provide a recommendation for updating the default system settings in accordance with one embodiment. In this embodiment, the system trending step 54 includes identifying the exam protocols that were utilized together in each set of parameter data that corresponds to a separate imaging operation (block 60). Further, the system trending step 54 includes comparing these identified exam protocol groupings across the parameter data sets (block 62) and, based on this comparison, identifying the frequently occurring groupings (block 64). For example, the method may identify that an operator frequently groups acquisition of chest images with abdominal images. Still further, it should be noted that different frequency thresholds may be developed for different imaging systems. For example, in some embodiments, a grouping may need to occur in greater than approximately 50%, 60%, 70% or 80% of the parameter sets for the grouping to be identified as “frequently occurring.” For further example, in other embodiments, the frequency threshold may be based on a number of times a grouping occurs, without regard to the percentage of data sets in which it occurs. Indeed, a variety of suitable frequency thresholds may be established by an operator upon setup of the imaging system.

Once the frequently occurring groups have been identified in accordance with the frequency thresholds for the given system, favorite groupings may be recommended or implemented (block 66). For example, if a particular grouping of exam protocols occurs often enough across the parameter data sets, that grouping may be recommended as a favorite group. A favorite group may appear as a new button or option for the operator to choose, for example, on a user interface of the imaging system. In this way, once a grouping is established as a favorite group, the operator may select the favorite group without selecting each exam protocol that is included in the favorite group. The foregoing feature may offer the advantage of reducing setup time associated with the imaging system since previously utilized settings that are frequently employed may be stored as default settings for future use.

In additional to trending of operator selected groupings of imaging exam protocols, trending of parameters within the imaging protocols or within steps of these protocols may also be performed in presently contemplated embodiments. In particular, FIG. 4 illustrates a method 68 for system trending of global parameters of a protocol to provide a recommendation for updating the default system settings in accordance with one embodiment. In this embodiment, the trending process 54 includes identifying the global parameters utilized in each received parameter data set (block 70) and comparing these global parameters across data sets for each exam type (block 72). Here again, the frequently occurring changes from the default values for each of the global parameters are identified (block 74) and recommendations for updates to the default values for the global parameters are recommended or implemented (block 76).

For example, in one embodiment, the method 68 may be utilized to identify global parameters of a protocol that are frequently changed for a particular type of patient, such as a pediatric patient. Subsequently, for future pediatric exams, the global parameter value may be altered from the default value in accordance with previously chosen selections. For further example, in another embodiment, global setup parameters for a particular type of exam, for example, a field of view for a fluoroscopy exam, may be trended to provide update recommendations.

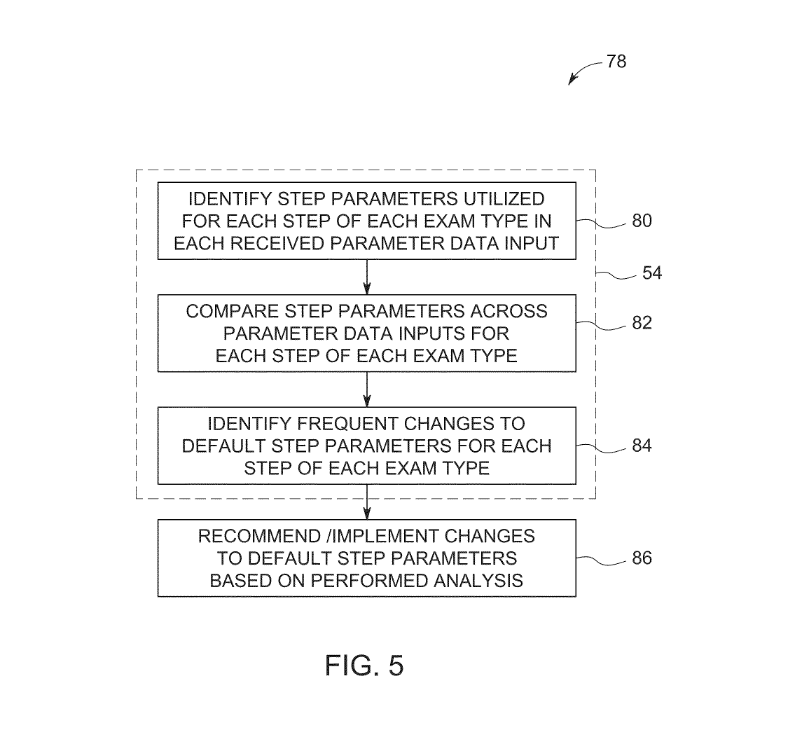

Still further, FIG. 5 illustrates a method 78 for system trending of step parameters specific to a step of an imaging protocol in accordance with one embodiment. For example, step parameters may include the kilovoltage peak or amperage of a step of a radiographic procedure. Here again, the method 78 includes identifying the step parameters selected by the operator for each step of each exam type included in the parameter data sets (block 80), comparing the identified step parameters (block 82), and identifying frequently occurring changes to the current default setting for each step parameter (block 84). The method 78 further includes updating or recommending updates to the step parameters that are frequently altered from their respective default values by the operator (block 86).

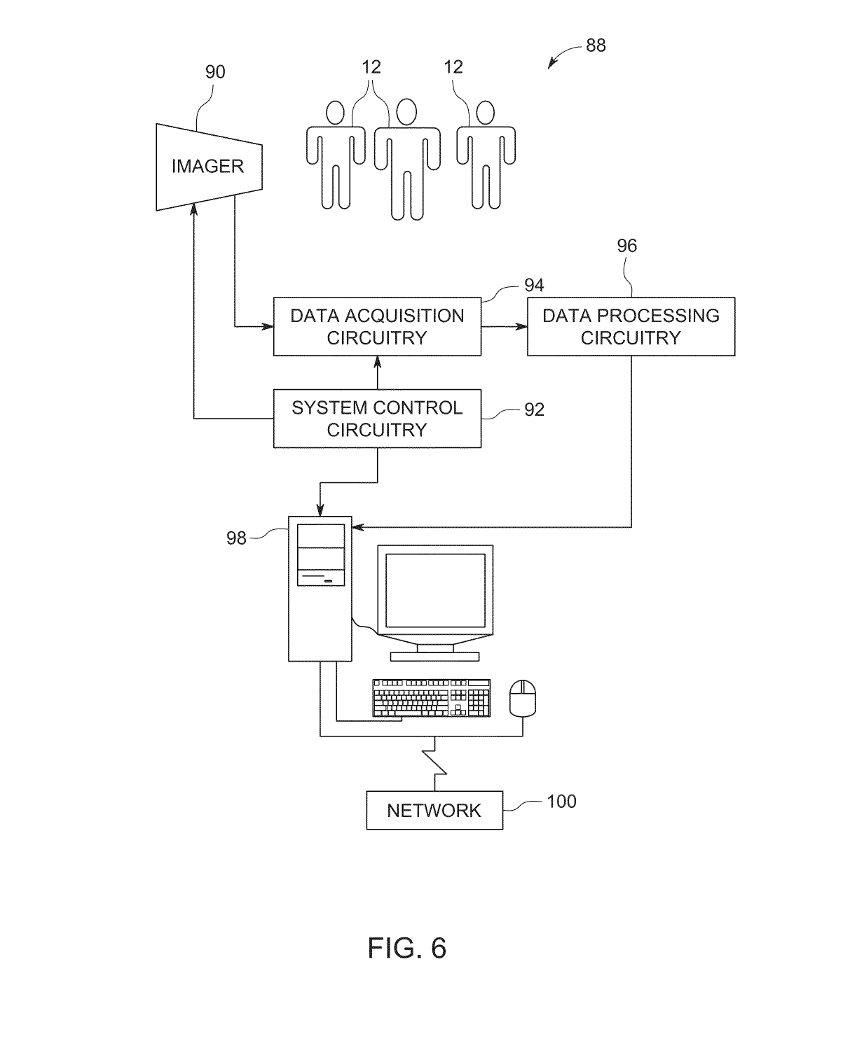

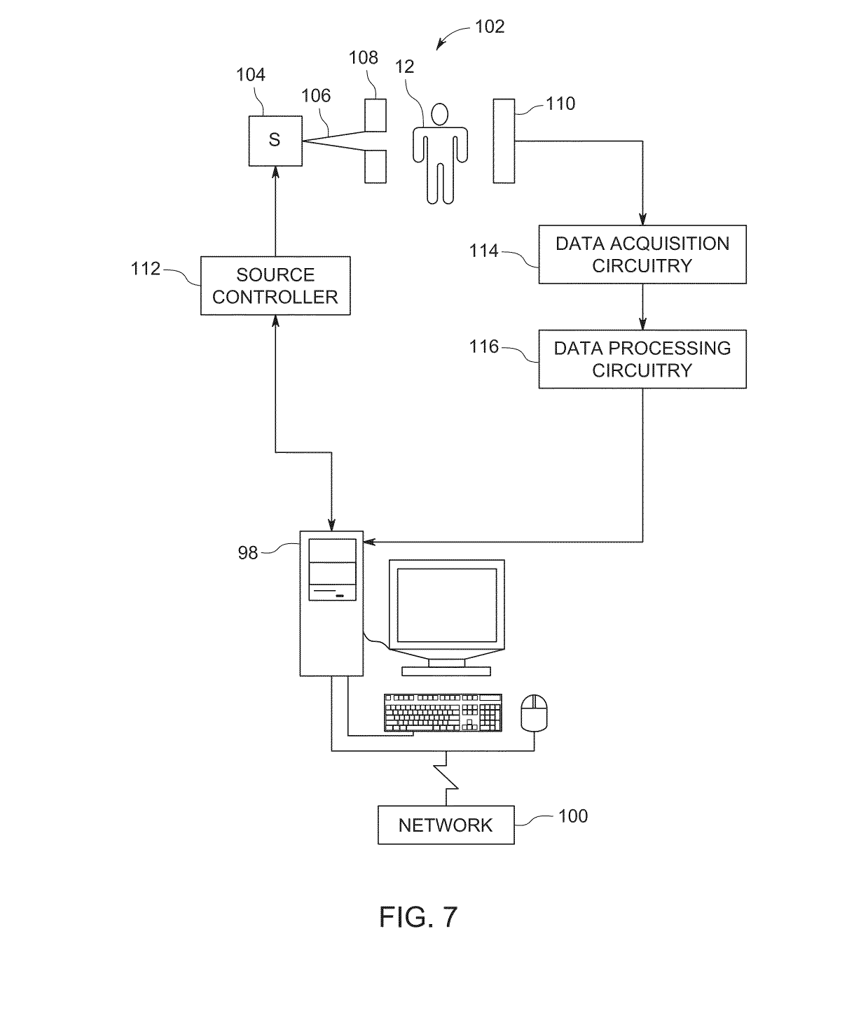

It should be noted that the previously described methods for monitoring and trending of parameters and groupings selected by an operator may be employed in a variety of types of imaging systems and are compatible with many imaging techniques. To that end, FIG. 6 provides a general overview of an exemplary imaging system 88 that may employ the described parameter monitoring and trending methods. The imaging system 88 includes an imager 90 that detects imaging signals and converts the signals to useful data. As described more fully below with respect to the particular modalities presented in FIGS. 7-10, the imager 90 may operate in accordance with various physical principles for creating the image data. In general, however, image data indicative of regions of interest in a patient are created by the imager either in a conventional support, such as photographic film, or in a digital medium.

The imager 90 operates under the control of system control circuitry 92. The system control circuitry 92 may include a wide range of circuits, such as radiation source control circuits, timing circuits, circuits for coordinating data acquisition in conjunction with patient or table of movements, circuits for controlling the position of radiation or other sources and of detectors, and so forth. The imager 90, following acquisition of the image data or signals in accordance with operator selected parameters, may process the signals, such as for conversion to digital values, and forward the image data and/or the parameter data to data acquisition circuitry 94. In the case of analog media, such as photographic film, the data acquisition system may generally include supports for the film, as well as equipment for developing the film and producing hard copies that may be subsequently digitized. For digital systems, the data acquisition circuitry 94 may perform a wide range of initial processing functions, such as adjustment of digital dynamic ranges, smoothing or sharpening of data, as well as compiling of data streams and files, where desired. The data is then transferred to data processing circuitry 96 where additional processing and analysis are performed. For conventional media such as photographic film, the data processing system may apply textual information to films, as well as attach certain notes or patient-identifying information. For the various digital imaging systems available, the data processing circuitry performs substantial analyses of data, ordering of data, sharpening, smoothing, feature recognition, and so forth.

Further, in particular embodiments, the data processing circuitry 96 may be associated with memory suitable for storing portions of the received data. That is, the processing circuitry 96 may either include its own memory, or may be associated with external memory, such as for storing algorithms and instructions executed by the processing circuitry during operation, as well as image data and/or parameter data on which the processing is performed. Furthermore, the data processing circuitry 96 may perform processing algorithms that facilitate a comparison of one or more parameters across received parameter data sets. In certain embodiments, the processing circuitry 50 may store the acquired parameter data sets corresponding to the operator selections for the imaging operations on the memory. The memory may be a removable form of memory, such as a USB flash drive or an SD card, or the memory may include volatile or non-volatile memory, such as read only memory (ROM), random access memory (RAM), magnetic storage memory, optical storage memory, or a combination thereof.

Ultimately, the image data and/or the parameter data is forwarded to some type of operator interface 98 for viewing and analysis. While operations may be performed on the image data and/or the parameter data prior to viewing, the operator interface 98 may facilitate viewing of reconstructed images based upon the image data collected. Additionally, the operator interface 98 may provide an interface for the operator to alter one or more default parameter or protocol settings in accordance with operator preferences. Still further, once a recommendation as to an update in the default parameter settings and/or protocol groupings has been developed by the data processing circuitry 96, the operator interface 98 may facilitate communication of these recommendations to the operator.

The image data and/or the parameter data as well as one or more update recommendations developed by the processing circuitry 96 may also be transferred to remote locations, such as via a network 100. It should also be noted that the operator interface 98 enables control of the imaging system, typically by interfacing with the system control circuitry 92. Moreover, it should also be noted that more than a single operator interface 98 may be provided. Accordingly, an imaging scanner or station may include an interface which permits regulation of the parameters involved in the image data acquisition procedure, whereas a different operator interface may be provided for manipulating, enhancing, and viewing resulting reconstructed images.

FIGS. 7-10 illustrate particular embodiments of imaging modalities that may be utilized with the foregoing methods to monitor and trend imaging parameters during system usage. Specifically, FIG. 7 is diagrammatical overview of an exemplary digital X-ray system 102 that may be employed in accordance with a presently disclosed embodiment. The illustrated X-ray system 102 includes a radiation source 104, typically an X-ray tube, designed to emit a beam 106 of radiation. The radiation may be conditioned or adjusted, typically by adjustment of parameters of the source 104, such as the type of target, the input power level, and the filter type. The resulting radiation beam 106 is typically directed through a collimator 108 that determines the extent and shape of the beam directed toward the patient 12. A portion of the patient 12 corresponding to a region of interest is placed in the path of beam 106, and the beam impacts a digital detector 110.

The detector 110, which typically includes a matrix of pixels, encodes intensities of radiation impacting various locations in the matrix. A scintillator converts the high energy X-ray radiation to lower energy photons that are detected by photodiodes within the detector. The X-ray radiation is attenuated by tissues within the patient, such that the pixels identify various levels of attenuation resulting in various intensity levels that will form the basis for an ultimate reconstructed image.

As before, control circuitry and data acquisition circuitry are provided for regulating the image acquisition process in accordance with operator selections and for detecting and processing the resulting image data and parameter data for each operation. In the illustrated embodiment, a source controller 112 is provided for regulating operation of the radiation source 104. Other control circuitry may be provided for controllable aspects of the system, such as a table position, radiation source position, and so forth. Data acquisition circuitry 114 is coupled to the detector 110 and permits readout of the charge on the photodetectors following an exposure. In general, charge on the photodetectors is depleted by the impacting radiation, and the photodetectors are recharged sequentially to measure the depletion. The readout circuitry may include circuitry for systematically reading rows and columns of the photodetectors corresponding to the pixel locations of the image matrix. The resulting signals are then digitized by the data acquisition circuitry 114 and forwarded to data processing circuitry 116.

The data processing circuitry 116 may perform a range of operations, including adjustment for offsets, gains, and the like in the digital image data, as well as various imaging enhancement functions. Additionally, the processing circuitry 116 may perform an analysis on multiple parameter data sets to trend the usage over a series of imaging operations. The resulting data is then forwarded to an operator interface or storage device for short or long-term storage. The images reconstructed based upon the data may be displayed on the operator interface, or may be forwarded to other locations, such as via a network 100 for viewing. Also, the recommendations for updates to one or more default parameter values or protocol settings may similarly be transferred to the operator interface 98 for acceptance or rejection by the operator.

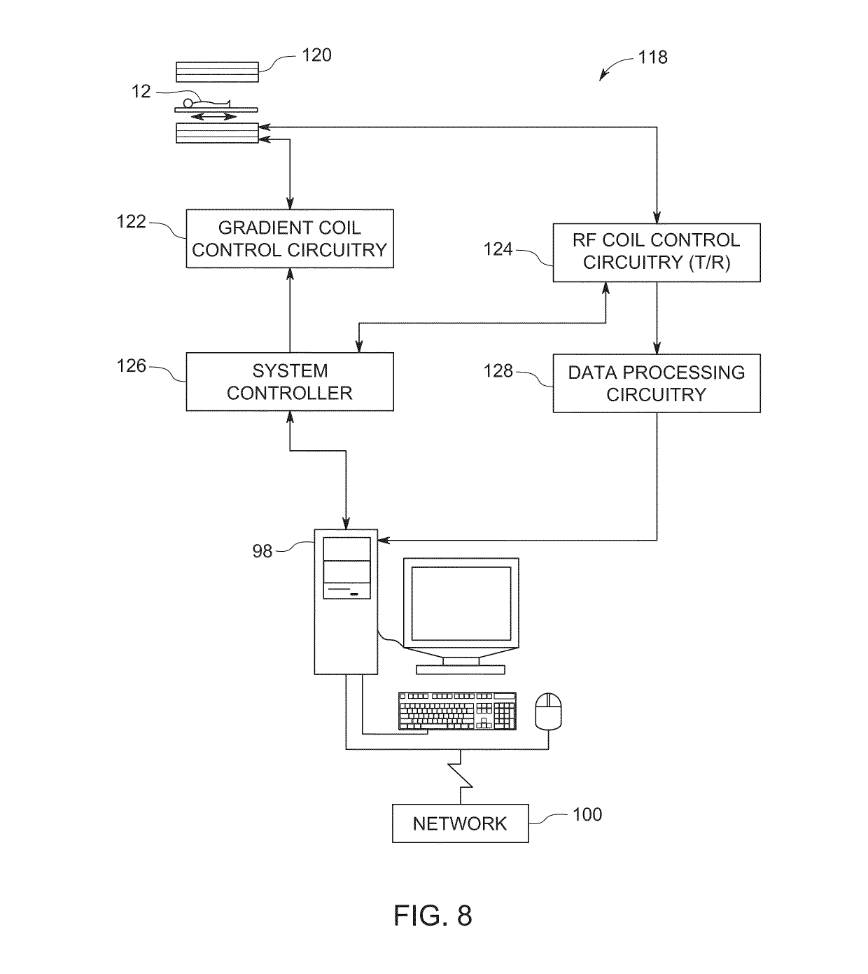

FIG. 8 is an overview of an exemplary magnetic resonance imaging system 118 that may be employed in accordance with a presently disclosed embodiment. The system 118 includes a scanner 120 in which a patient is positioned for acquisition of image data. The scanner 120 generally includes a primary magnet for generating a magnetic field that influences gyromagnetic materials within the patient's body. As the gyromagnetic material, typically water and metabolites, attempts to align with the magnetic field, gradient coils produce additional magnetic fields which are orthogonally oriented with respect to one another. The gradient fields effectively select a slice of tissue through the patient for imaging, and encode the gyromagnetic materials within the slice in accordance with phase and frequency of their rotation. A radio-frequency (RF) coil in the scanner generates high frequency pulses to excite the gyromagnetic material and, as the material attempts to realign itself with the magnetic fields, magnetic resonance signals are emitted and collected by the radio-frequency coil.

The scanner 120 is coupled to gradient coil control circuitry 122 and to RF coil control circuitry 124. The gradient coil control circuitry 122 permits regulation of various pulse sequences which define imaging or examination methodologies used to generate the image data. Pulse sequence descriptions implemented via the gradient coil control circuitry 122 are designed to image specific slices and anatomies, as well as to permit specific imaging of moving tissue, such as blood, and defusing materials. The pulse sequences may allow for imaging of multiple slices sequentially, such as for analysis of various organs or features, as well as for three-dimensional image reconstruction. The RF coil control circuitry 124 permits application of pulses to the RF excitation coil and serves to receive and partially process the resulting detected MR signals. It should also be noted that a range of RF coil structures may be employed for specific anatomies and purposes. In addition, a single RF coil may be used for transmission of the RF pulses, with a different coil serving to receive the resulting signals.

The gradient and RF coil control circuitry function under the direction of a system controller 126. The system controller 126 implements pulse sequence descriptions which define the image data acquisition process. The system controller will generally permit some amount of adaptation or configuration of the examination sequence by means of the operator interface 98. That is, the operator may utilize the operator interface 98 to evaluate and, if necessary, alter one or more default parameters that define operation of the imaging system 118.

Data processing circuitry 128 receives the detected MR signals and processes the signals to obtain data for reconstruction. In general, the data processing circuitry 28 digitizes the received signals, and performs a two-dimensional fast Fourier transform on the signals to decode specific locations in the selected slice from which the MR signals originated. The resulting information provides an indication of the intensity of MR signals originating at various locations or volume elements (voxels) in the slice. Each voxel may then be converted to a pixel intensity in image data for reconstruction.

The data processing circuitry 128 may perform a wide range of other image data processing functions as well, such as for image enhancement, dynamic range adjustment, intensity adjustments, smoothing, sharpening, and so forth. Further, the data processing circuitry 128 may be adapted to receive and process parameter data for a series of performed imaging operations. That is, the processing circuitry 128 may monitor and trend the changes the operator makes to default imaging settings in order to provide one or more update recommendations. The processed image data and the update recommendations are typically forwarded to the operator interface 98 for viewing.

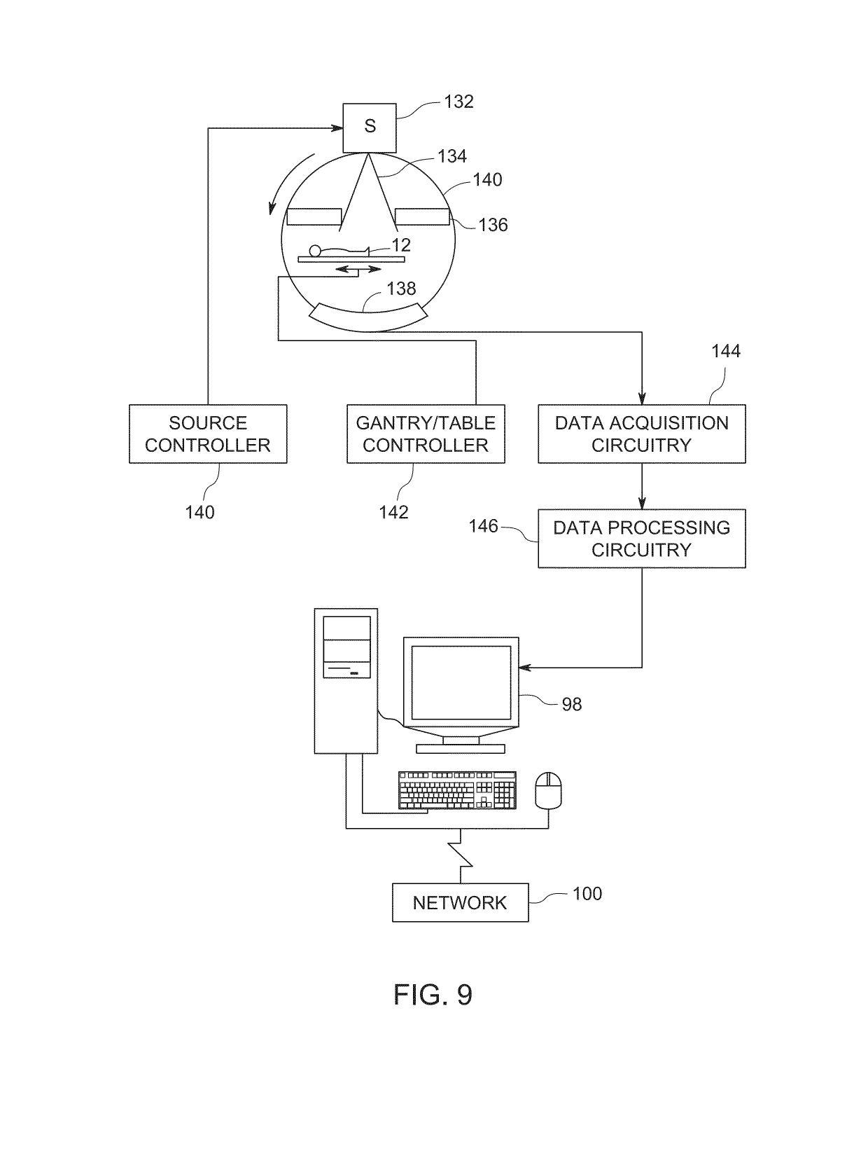

FIG. 9 is a diagrammatical overview of an exemplary computed tomography (CT) imaging system 130 that may be employed in a presently disclosed embodiment. The CT imaging system 130 includes a radiation source 132 which is configured to generate X-ray radiation in a fan-shaped beam 134. A collimator 136 defines limits of the radiation beam. The radiation beam 134 is directed toward a curved detector 138 made up of an array of photodiodes and transistors which permit readout of charges of the diodes depleted by impact of the radiation from the source 132. The radiation source, the collimator and the detector are mounted on a rotating gantry 140 which enables them to be rapidly rotated (such as at speeds of two rotations per second).

During an examination sequence, as the source and detector are rotated, a series of view frames are generated at angularly-displaced locations around the patient 12 positioned within the gantry. A number of view frames (e.g. between 500 and 1000) are collected for each rotation, and a number of rotations may be made, such as in a helical pattern as the patient is slowly moved along the axial direction of the system. For each view frame, data is collected from individual pixel locations of the detector to generate a large volume of discrete data.

A source controller 140 regulates operation of the radiation source 132, while a gantry/table controller 142 regulates rotation of the gantry and control of movement of the patient. Data collected by the detector 138 is digitized and forwarded to data acquisition circuitry 144. The data acquisition circuitry 144 may perform initial processing of the image data and the parameter data, such as for generation of a data file. The data file may incorporate other useful information, such as relating to cardiac cycles, positions within the system at specific times, and so forth. Data processing circuitry 146 then receives the data and performs a wide range of data manipulation and computations. For example, as described in detail above, the processing circuitry 146 may identify one or more trends in the imaging parameters or protocols implemented during operation of the system 130. Further, update recommendations based on this trending may be developed by the processing circuitry 146 and made available to an operator, such as at an operator interface 98 and/or may be transmitted remotely via a network connection 100.

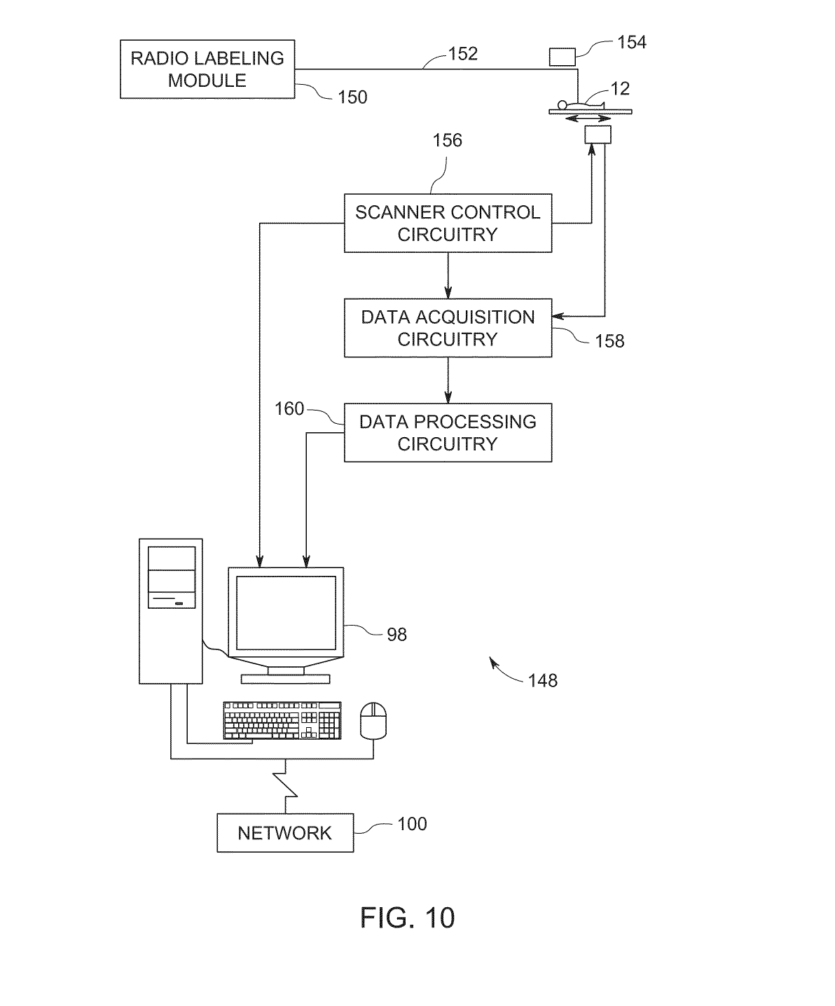

FIG. 10 illustrates an exemplary positron emission tomography (PET) imaging system 148 that may be employed in accordance with an embodiment. The PET imaging system 148 includes a radio-labeling module 150 which is sometimes referred to as a cyclotron. The cyclotron is adapted to prepare certain tagged or radio-labeled materials, such as glucose, with a radioactive substance. The radioactive substance is then injected into a patient 12, as indicated by reference numeral 152. The patient 14 is then placed in a PET scanner 154. The scanner 154 detects emissions from the tagged substance as its radioactivity decays within the body of the patient 14. In particular, positrons, sometimes referred to as positive electrons, are emitted by the material as the radioactive nuclide level decays. The positrons travel short distances and eventually combine with electrons resulting in emission of a pair of gamma rays. Photomultiplier-scintillator detectors within the scanner detect the gamma rays and produce signals based upon the detected radiation.

The scanner 154 operates under the control of scanner control circuitry 156, itself regulated by an operator interface 98. In most PET scans, the entire body of the patient is scanned, and signals detected from the gamma radiation are forwarded to data acquisition circuitry 158. The particular intensity and location of the radiation can be identified by data processing circuitry 160, and reconstructed images may be formulated and viewed on operator interface 98, or the raw or processed data may be stored for later image enhancement, analysis, and viewing. The images, or image data, may also be transmitted to remote locations via a network link 100. Similarly, the processing circuitry 160 also receives signals encoding the parameter and protocol data and processes these signals to identify one or more update recommendations to the operator via the interface 98.

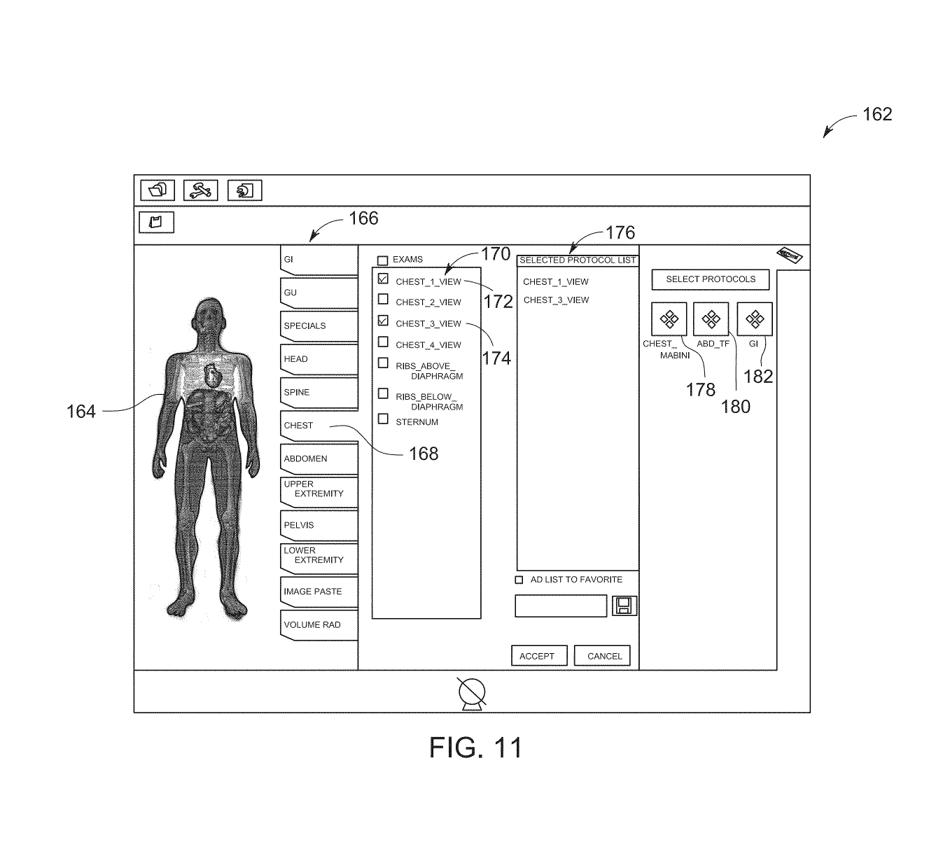

One embodiment of an exemplary operator interface 162 that may be utilized by an operator to select imaging parameters and exam protocols is shown in FIG. 11. In the illustrated embodiment, the interface 162 includes a diagram 164 of patient anatomy and a list 166 corresponding to regions of interest available for imaging with the associated imaging system. In the depicted view, a chest tab 168 has been selected by the operator. Accordingly, a list 170 of exam protocols available for selection and corresponding to the chest region of the anatomy 164 are shown. In the illustrated embodiment, the operator has chosen a first chest view 172 and a third chest view 174 for inclusion in the protocol grouping 176 for the given imaging operation.

In some embodiments, the protocol grouping 176 may be exported as part of the parameter data set for the given imaging operation. The processing circuitry may then compare the protocol grouping 176 to other protocol groupings established for other imaging operations to identify exam protocols (e.g., 172 and 174) that are frequently grouped together in different imaging operations. Once identified, the frequently grouped exam protocols may be recommended to the operator for establishment as a favorite grouping, for example, favorite groupings 178, 180, and 182. Once a list of exam protocols is established as a favorite grouping, the operator may select the desired favorite grouping button (e.g., 178, 180, or 182) without manually selecting each exam protocol when the operator wishes to repeat a similar imaging procedure. Again, the foregoing feature may reduce the amount of time necessary for an operator to set up the imaging system for data collection, thus increasing operator efficiency.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A medical imaging analysis method, comprising:
receiving parameter data from an imaging component, wherein the parameter data corresponds to at least two imaging operations and encodes at least two parameter sets corresponding to the at least two imaging operations; and
comparing the at least two parameter sets to identify a grouping that repeats between the parameter sets a number of times that exceeds a first threshold, an implemented change to a default parameter that repeats between the parameter sets a number of times that exceeds a second threshold, or a combination thereof.

2. The method of claim 1, comprising alerting an operator to a recommended grouping of a series of imaging protocols based on the grouping that repeats beyond the first threshold.

3. The method of claim 1, comprising informing an operator of a recommended change to the default parameter based on the change that repeats beyond the second threshold.

4. The method of claim 1, wherein the imaging operations comprise at least one of a digital x-ray imaging operation, a magnetic resonance imaging operation, a computed tomography operation, a positron emission tomography operation, and an ultrasound operation.

5. The method of claim 1, wherein the grouping comprises a selected grouping of imaging exam protocols that each corresponds to a view of a feature of a patient's anatomy.

6. The method of claim 5, wherein the default parameter comprises a protocol parameter specific to one of the imaging exam protocols.

7. The method of claim 6, wherein the protocol parameter comprises a starting magnification, a starting density, a starting dose level, or a combination thereof.

8. A medical imaging system, comprising:
an imager configured to generate image data indicative of a region of interest in a patient;
an operator interface configured to receive one or more operator selections corresponding to parameters of an imaging operation to be performed by the imager;
control circuitry coupled to the imager and the operator interface and configured to control the imager in accordance with the operator selections to acquire signals that may be converted to the image data; and
data processing circuitry configured to receive a parameter set containing the operator selections from the operator interface and to analyze the received parameter set via comparison with a previously received parameter set to identify the frequency of one or more changes to one or more default parameters.

9. The system of claim 8, wherein the data processing circuitry is further configured to communicate with the operator interface to recommend one or more alterations to the default parameters when the frequency of the changes to the default parameters exceeds a threshold value.

10. The system of claim 8, wherein the one or more default parameters comprise a starting field of view, a starting magnification, a starting density, a starting dose level, or a combination thereof.

11. The system of claim 8, wherein the imager comprises at least one of an x-ray imaging device, a positron emission tomography device, an ultrasound imaging device, and a magnetic resonance imaging device.

12. The system of claim 8, wherein the data processing circuitry is configured to receive the image data from the imager and to convert the image data to a visual representation of the region of interest of the patient for display on a panel of the operator interface.

13. A medical imaging system, comprising:
an operator interface configured to receive operator selections corresponding to a desired exam protocol grouping for an imaging operation to be performed by a medical imaging device; and
data processing circuitry configured to receive a parameter set containing the operator selections from the operator interface, to compare the desired exam protocol grouping with at least one previously received exam protocol grouping to identify one or more protocol groupings that occur at a frequency exceeding a threshold, and to recommend establishment of the identified protocol groupings as favorite groups.

14. The system of claim 13, wherein the data processing circuitry is configured to communicate with the operator interface to establish display buttons on the operator interface that correspond to each of the favorite groups.

15. The system of claim 13, wherein the desired exam protocol grouping includes one or more protocol selections corresponding to regions of interest in a patient.

16. The system of claim 13, comprising an imager configured to operate in accordance with the operator selections to generate image data indicative of a region of interest in a patient.

17. The system of claim 16, wherein the imager comprises at least one of an x-ray imaging device, a positron emission tomography device, an ultrasound imaging device, and a magnetic resonance imaging device.

18. The system of claim 13, wherein the operator interface is further configured to receive operator selections corresponding to values for parameters of each of the desired exam protocols in the desired exam protocol grouping.

19. The system of claim 18, wherein the data processing circuitry is configured to compare the operator selected parameter values with at least one previously received operator selected parameter value to identify one or more changes to a default value for the parameter that occur at a frequency exceeding a second threshold.

20. The system of claim 19, wherein the data processing circuitry is configured to recommend a change to the default value for the identified parameter by alerting the operator via the operator interface.
